(19) United States
(12) Patent Application Publication
(10) Pub. No.: US 2021/0298655 A1
(43) Pub. Date: Sep. 30, 2021

(54) ELECTRODE SENSOR AND METHOD FOR FABRICATING SAME

(71) Applicant: ELECTRONICS AND TELECOMMUNICATIONS RESEARCH INSTITUTE, Daejeon (KR)

(72) Inventors: Yong Hee KIM, Daejeon (KR); Sang Don JUNG, Daejeon (KR)

(73) Assignee: ELECTRONICS AND TELECOMMUNICATIONS RESEARCH INSTITUTE, Daejeon (KR)

(21) Appl. No.: 17/208,635

(22) Filed: Mar. 22, 2021

(30) Foreign Application Priority Data
Mar. 26, 2020 (KR) 10-2020-0036975

(51) Int. Cl. A61B 5/268

(52) U.S. Cl. CPC A61B 5/268; A61B 2562/125; A61B 2562/166

(57) ABSTRACT

Provided are an electrode sensor and a method for fabricating the same. According to embodiments, an electrode sensor includes: an organic substrate including a polymer; an organic insulating pattern which is disposed on the organic substrate and has an opening through which the organic substrate is exposed; a conductive layer which is disposed on the organic substrate and within the opening of the organic insulating pattern; and a passivation pattern which is disposed on the organic insulating pattern and through which a portion of the conductive layer is exposed. The conductive layer may include a conductive polymer, and the conductive layer may be coupled to the organic substrate by a covalent bond.

### CROSS-REFERENCE TO RELATED APPLICATIONS

This U.S. non-provisional patent application claims priority under 35 U.S.C. § 119 of Korean Patent Application No. 10-2020-0036975, filed on Mar. 26, 2020, the entire contents of which are hereby incorporated by reference.

### BACKGROUND

The present disclosure herein relates to an electrode sensor and a method for fabricating the same, and more particularly, to a neural electrode sensor and a method for fabricating the same.

Electrode sensors may include electrodes. Such an electrode may provide electrical stimulation to a detection target or may detect a signal. The electrode is implanted into a biological neural tissue or the like and used to record a neural signal or apply electrical stimulation.

The electrode may be easily attached to a living body as the elasticity thereof increases. A flexible electrode may have high potential for application in the field of biomedical application. For example, neural signals, biosignals, or chemical substances may be detected by using the flexible electrode. Also, the interest in a wearable electronic system or skintronics is increasing, and the flexible electrode may be used in the wearable electronic system or skintronics. Regarding a metal electrode, there has been a limitation that the metal electrode is separated from a substrate.

### SUMMARY

The present disclosure provides a flexible electrode sensor and a method for fabricating the same.

The present disclosure also provides an electrode sensor having improved durability and a method for fabricating same.

The object of the present disclosure is not limited to the aforesaid, but other objects not described herein will be clearly understood by those skilled in the art from following description.

The present disclosure relates to an electrode sensor and a method for fabricating the same. An embodiment of the inventive concept provides an electrode sensor including: an organic substrate including a polymer; an organic insulating pattern disposed on the organic substrate and has an opening, exposing the organic substrate; a conductive layer which is disposed on the organic substrate and in the opening of the organic insulating pattern; and a passivation pattern which is disposed on the organic insulating pattern and through which a portion of the conductive layer is exposed. The conductive layer may include a first polymer, and the conductive layer may be coupled to the organic substrate by a covalent bond.

In embodiments, the conductive layer may be coupled to the organic insulating pattern by a covalent bond.

In embodiments, the organic insulating pattern may be coupled to the organic substrate by a covalent bond.

In embodiments, the passivation pattern may be coupled to the organic insulating pattern by a covalent bond.

In embodiments, the conductive layer may be coupled to the passivation pattern by a covalent bond.

In embodiments, the conductive layer may include an electrode and a wire, and the wire may be connected to the electrode.

In embodiments, the passivation pattern may cover a top surface of the wire, wherein a top surface of the electrode is exposed by the passivation pattern.

In embodiments, the organic substrate may include a fluorine-containing polymer, and the organic insulating pattern may include a fluorine-containing polymer.

In embodiments, the passivation pattern may include a fluorine-containing polymer.

In embodiments, the conductive layer may include a different material from the organic insulating pattern.

In embodiments, the first polymer may include an insulating polymer, the conductive layer may further include a conductive material, and the conductive material may include a metal particle or a conductive carbon material.

In embodiments, the first polymer may include a conductive polymer.

In embodiments, the organic substrate, the organic insulating pattern, the conductive layer, and the passivation pattern may be flexible.

In an embodiment of the inventive concept, a method for fabricating an electrode sensor includes: forming an organic insulating pattern that has an opening on an organic substrate, wherein a top surface of the organic substrate is exposed by the opening; forming a preliminary conductive layer, which covers the exposed top surface of the organic substrate, by filling the opening of the organic insulating pattern with a polymer solution; and forming a conductive layer by performing an exposure process on the preliminary conductive layer and the organic insulating pattern, wherein the conductive layer is coupled to the organic substrate by a covalent bond, and the conductive layer includes first polymers.

In embodiments, the performing of the exposure process may further include forming a covalent bond between the conductive layer and the organic insulating pattern.

In embodiments, the method may further includes forming a radical on the exposed top surface of the organic substrate, by performing a plasma treatment process on the exposed top surface of the organic substrate and a top surface of the organic insulating pattern, wherein the forming of the preliminary conductive layer is performed after the plasma treatment process.

In embodiments, the method may further include: forming a passivation layer on the organic insulating pattern after the exposure process; and forming a covalent bond between the passivation layer and the organic insulating pattern by irradiating the passivation layer with ultraviolet light.

In embodiments, the method of claim may further include forming radicals on a top surface of the conductive layer and a top surface of the organic insulating pattern by performing a plasma treatment process on the conductive layer and the organic insulating pattern, wherein the forming of the passivation layer is performed after the plasma treatment process.

In embodiments, the irradiating of the passivation layer with the ultraviolet light may further include forming a covalent bond between the passivation layer and the conductive layer.

In embodiments, the performing of the exposure process may include forming photocrosslink bonds between the first polymers in the conductive layer.

### BRIEF DESCRIPTION OF THE FIGURES

The accompanying drawings are included to provide a further understanding of the inventive concept, and are incorporated in and constitute a part of this specification. The drawings illustrate embodiments of the inventive concept and, together with the description, serve to explain principles of the inventive concept. In the drawings:

FIG. 1A is a plan view illustrating an electrode sensor according to embodiments;

FIG. 1B is a cross-sectional view taken along line A-B of FIG. 1A; and

FIGS. 2A to 2M are views for explaining a method for fabricating an electrode sensor according to embodiments.

### DETAILED DESCRIPTION

Embodiments of the present disclosure will be described with reference to the accompanying drawings so as to sufficiently understand constitutions and effects of the present disclosure. The present disclosure may, however, be embodied in different forms and diversely modified, and should not be construed as limited to the embodiments set forth herein. Rather, these embodiments are provided so that this disclosure will be thorough and complete, and will fully convey the scope of the present disclosure to a person skilled in the art to which the present disclosure pertains. Those skilled in the art will understand that the inventive concept can be implemented in an appropriate environment.

The terms used in this specification are used only for explaining embodiments while not limiting the present disclosure. In this specification, the singular forms include the plural forms as well, unless the context clearly indicates otherwise. The meaning of ‘comprises’ and/or ‘comprising’ used in the specification does not exclude the presence or addition of one or more components, steps, operations, and/or elements other than the mentioned components, steps, operations, and/or elements.

In the specification, it will be understood that when a film (or layer) is referred to as being on another film (or layer) or substrate, it can be directly on another film (or layer) or substrate, or intervening elements (or layers) may be disposed therebetween.

Also, though terms like a first, a second, and a third are used to describe various regions and films (or layers) in various embodiments of the specification, the regions and films are not limited to these terms. These terms are used only to distinguish a predetermined region or film (or layer) from another region or film (or layer). Therefore, a layer referred to as a first layer in one embodiment can be referred to as a second layer in another embodiment. Each of embodiments described and exemplified herein also includes a complementary embodiment thereof. Like reference numerals refer to like elements throughout.

Unless otherwise defined, all terms used in embodiments of the inventive concept have the same meaning as commonly understood by one of ordinary skill in the art to which the present disclosure belongs.

Hereinafter, an electrode sensor according to an embodiment of the inventive concept will be described with reference to the accompanying drawings.

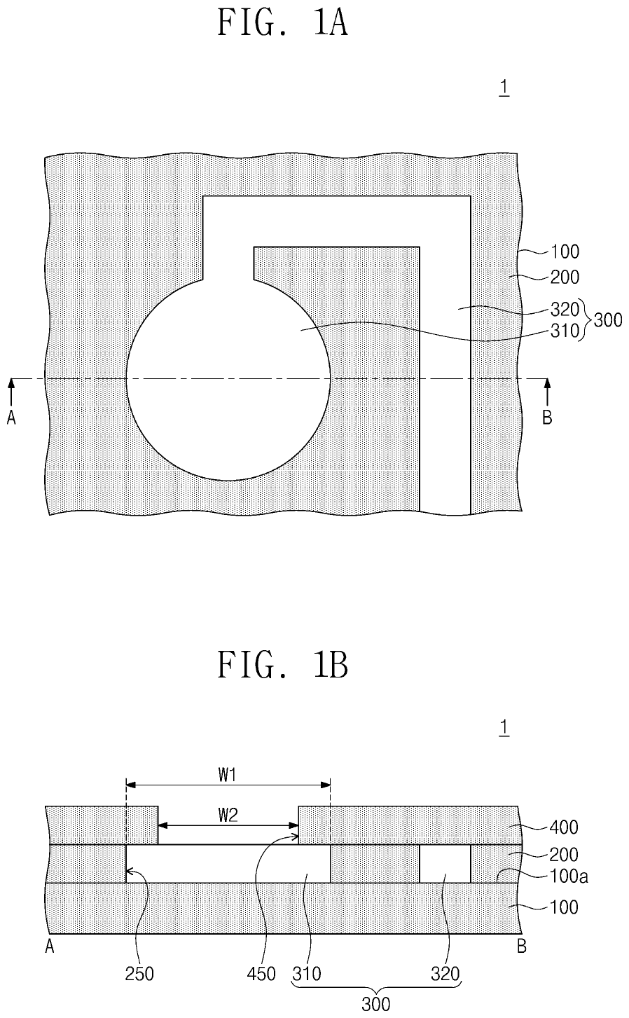

FIG. 1A is a plan view illustrating an electrode sensor according to embodiments. FIG. 1B is a cross-sectional view taken along line A-B of FIG. 1A.

Referring to FIGS. 1A and 1B, an electrode sensor 1 may include an organic substrate 100, an organic insulating pattern 200, a conductive layer 300, and a passivation pattern 400. The electrode sensor 1 may be flexible.

The organic substrate 100 may include a polymer and exhibit insulating characteristics. For example, the organic substrate 100 may include a fluorine-containing polymer, which may be a photocrosslinked polymer. In the specification, the fluorine-containing polymer may be a hydrofluorocarbon-based polymer and defined as having a plurality of carbon-fluorine (C—F) bonds. The fluorine-containing polymer may include perfluoropolyether (PFPE)-urethane acrylate. The organic substrate 100 may be flexible.

The organic insulating pattern 200 may be disposed on the organic substrate 100. The organic insulating pattern 200 may include a polymer. The organic insulating pattern 200 may include, for example, a fluorine-containing polymer which is photocrosslinked. An example of a material of the fluorine-containing polymer may be the same as that described above. The organic insulating pattern 200 may include the same polymer as the organic substrate 100 or a different polymer from the organic substrate 100. The organic insulating pattern 200 may be coupled to the organic substrate 100 by a covalent bond. For example, the covalent bond may be provided between a top surface 100a of the organic substrate 100 and a bottom surface of the organic insulating pattern 200. The covalent bond may include, for example, a crosslink bond between the polymer of the organic substrate 100 and the polymer of the organic insulating pattern 200. A coupling strength between the organic substrate 100 and the organic insulating pattern 200 is increased due to the covalent bond, and thus the durability of the electrode sensor 1 may be improved. The interfaces between the organic substrate 100 and the organic insulating pattern 200 may not be distinguished, but the embodiment of the inventive concept is not limited thereto. The organic insulating pattern 200 may have a first opening 250 therein. The top surface 100a of the organic substrate 100 may be exposed by the first opening 250. The organic insulating pattern 200 may be flexible.

The conductive layer 300 is formed within the first opening 250 of the organic insulating pattern 200 and may cover the top surface 100a of the exposed organic substrate 100. The conductive layer 300 may not extend to the top surface of the organic insulating pattern 200. According to an embodiment, the conductive layer 300 may include a first polymer and may exhibit conductive characteristics. The first polymer may include crosslinked polymers. The conductive layer 300 includes the first polymer and thus may be flexible. In an embodiment, the first polymer may be an insulating polymer, and the conductive layer 300 may further include conductive materials. The insulating polymer may include, for example, polydimethylsiloxane (PDMS). The conductive materials may be provided within the insulating polymer. The conductive materials may include metal materials and/or a conductive carbon material. The metal materials may include, for example, metal nanoparticles. The metal materials may include gold, platinum, iridium, copper, aluminum, and/or tungsten. The conductive carbon material may include, for example, a carbon nanotube. The conductive layer 300 may exhibit the conductive characteristics by the conductive materials. According to another embodiment, the first polymer may include a conductive polymer. The conductive polymer may include, for example, doped polyethylene, polypyrrole, and/or polythiophene. The conductive layer 300 may include a material different from those of the organic substrate 100, the organic insulating pattern 200, and the passivation pattern 400. The different material may be the conductive materials or conductive polymer described above.

The conductive layer 300 may include an electrode 310 and a wire 320. The electrode 310 may be a neural electrode. The neural electrode may be used to provide electrical stimulation to a biomaterial, measure an electrical signal (for example, a neural signal) of the biomaterial, or record the electrical signal. The biomaterial may include nerve cells and/or nerve tissues. The electrode 310 may have a circular shape as illustrated in FIG. 1A. In another example, the shape of the electrode 310 may be variously changed to a polygon, an oval, and/or the like. Although not illustrated, a plurality of electrodes 310 may be provided to form an electrode array.

The wire 320 may be connected to the electrode 310 as illustrated in FIG. 1A. The wire 320 may be an electrical interconnection. The wire 320 may be formed through the same process as the electrode 310. For example, the wire 320 may be connected to the electrode 310 without a boundary surface and may include the same material as the electrode 310. A thickness of the wire 320 may be substantially the same as the thickness of the electrode 310. The wire 320 may transmit an electrical signal generated in the electrode 310 to the outside or may transmit an external electrical signal to the electrode 310. The planar shape and arrangement of the wire 320 may be variously modified.

The conductive layer 300 may have electrical conductivity of about 10⁻⁷ Scm⁻¹ or higher. The conductive layer 300 has conductive characteristics, and this may mean that the conductive layer 300 has electrical conductivity of about 10⁻⁷ Scm⁻¹ or higher. When the conductivity of the conductive layer 300 is less than about 10⁻⁷ Scm⁻¹, it may be difficult for the conductive layer 300 to function as the wire 320 or the electrode 310.

The conductive layer 300 may be coupled to the organic substrate 100 by a covalent bond. The covalent bond may be provided between the top surface 100a of the organic substrate 100 and the bottom surface of the conductive layer 300. The covalent bond may include, for example, a crosslink bond between the polymer of the organic substrate 100 and the polymer of the conductive layer 300. A coupling strength between the organic substrate 100 and the conductive layer 300 may be increased due to the covalent bond. The conductive layer 300 may be coupled to the organic insulating pattern 200 by a covalent bond. The covalent bond may be provided between a side surface of the conductive layer 300 and a side surface of the organic insulating pattern 200. The covalent bond may include, for example, a crosslink bond between the polymer of the organic insulating pattern 200 and the polymer of the conductive layer 300. A coupling strength between the organic insulating pattern 200 and the conductive layer 300 is increased due to the covalent bond, and thus the durability of the electrode sensor 1 may be improved.

The passivation pattern 400 may be disposed on the top surface of the organic insulating pattern 200 and the top surface of the conductive layer 300. For example, the passivation pattern 400 covers the top surface of the organic insulating pattern 200 and the top surface of the wire 320, and thus the top surface of the wire 320 may not be exposed to the outside. The passivation pattern 400 has a second opening 450, and a portion of the top surface of the conductive layer 300 may be exposed by the second opening 450. At least a portion of the top surface of the electrode 310 may be exposed by the second opening 450 of the passivation pattern 400. For example, a central area of the top surface of the electrode 310 may be exposed by the second opening 450 of the passivation pattern 400. The top surface of the exposed electrode 310 may function as a sensing surface. A width W2 of the second opening 450 may be equal to or less than a width W1 of the electrode 310. The passivation pattern 400 may further extend to the top surface on an edge portion of the electrode 310. On the contrary, the top surface of the edge portion of the electrode 310 may be exposed by the passivation pattern 400.

The passivation pattern 400 may include a fluorine-containing polymer which is photocrosslinked and may have insulating characteristics. The conductive layer 300 may include the same polymer as the organic substrate 100 or a different polymer from the organic substrate 100. The conductive layer 300 may include the same polymer as the organic insulating pattern 200 or a different polymer from the organic insulating pattern 200.

The passivation pattern 400 may be coupled to the organic insulating pattern 200 by a covalent bond. The covalent bond may be provided between the top surface of the organic insulating pattern 200 and the bottom surface of the passivation pattern 400. The covalent bond may include, for example, a crosslink bond between the polymer of the organic insulating pattern 200 and the polymer of the passivation pattern 400. A coupling strength between the passivation pattern 400 and the organic insulating pattern 200 may be increased due to the covalent bond. The passivation pattern 400 may be coupled to the conductive layer 300 by a covalent bond. The covalent bond may be provided between the top surface of the wire 320 and the bottom surface of the passivation pattern 400. When the passivation pattern 400 extends to the top surface of the electrode 310, a covalent bond may be further provided between the top surface of the electrode 310 and the bottom surface of the passivation pattern 400. The covalent bond may include, for example, a crosslink bond between the polymer of the conductive layer 300 and the polymer of the passivation pattern 400. A coupling strength between the passivation pattern 400 and the conductive layer 300 may be increased due to the covalent bond. The interfaces between the passivation pattern 400 and the organic insulating pattern 200 may not be distinguished, but the embodiment of the inventive concept is not limited thereto.

In a case where the wire 320 and the electrode 310 include a metal layer formed by deposition or electroplating, an electrical short circuit may occur within the wire 320 or the electrode 310 when the wire 320 or the electrode 310 is bent. Also, when the electrode sensor 1 continues to operate, the wire 320 or the electrode 310 may be separated from an insulating component. The insulating component may be the organic substrate 100, the organic insulating pattern 200, or the passivation pattern 400. According to embodiments, the conductive layer 300 includes a polymer, and thus the conductive layer 300 may have improved durability and flexibility. According to embodiments, the organic substrate 100, the organic insulating pattern 200, the conductive layer 300, and the passivation pattern 400 are flexible, the electrode sensor 1 may become more flexible. Accordingly, the electrode sensor 1 may be easily attached to a living body. The electrode sensor 1 may have great potential for application.

The fluorine-containing polymer may be chemically stable. According to embodiments, the organic substrate 100, the organic insulating pattern 200, and the passivation pattern 400 include the fluorine-containing polymer and thus may have high durability against chemical substances. The chemical substances may include strong acids, strong bases, or solvents. The electrode sensor 1 may be used in various environments. The electrode sensor 1 may have implant stability and durability in the living body.

Hereinafter, a method for fabricating an electrode sensor according to an embodiment of the inventive concept will be described with reference to the accompanying drawings.

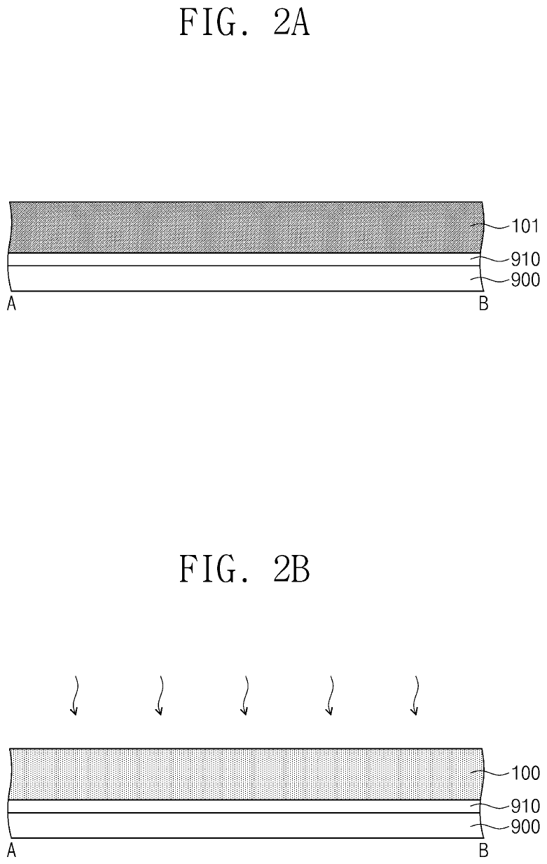

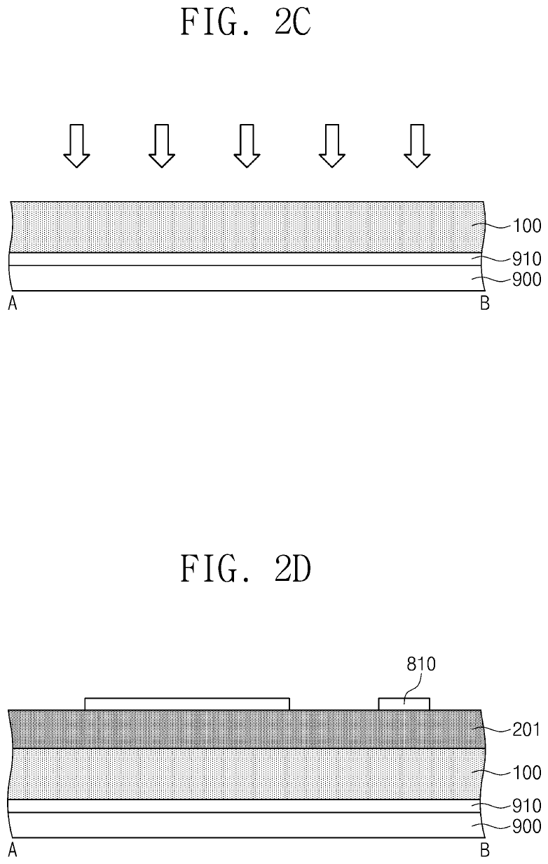

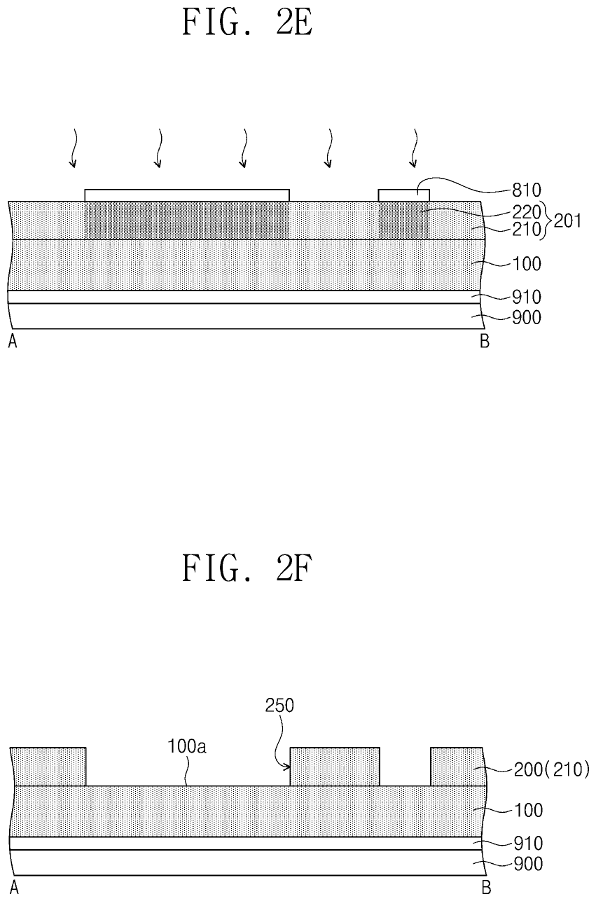

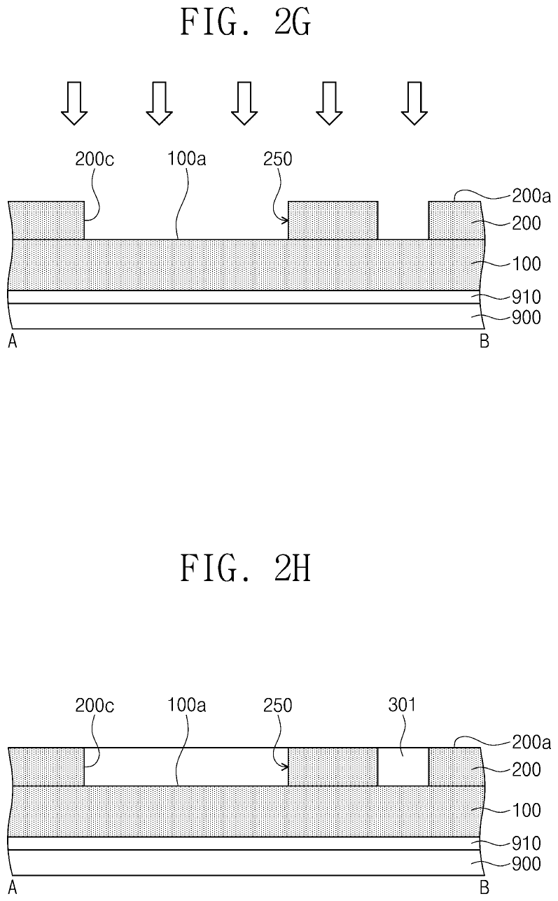

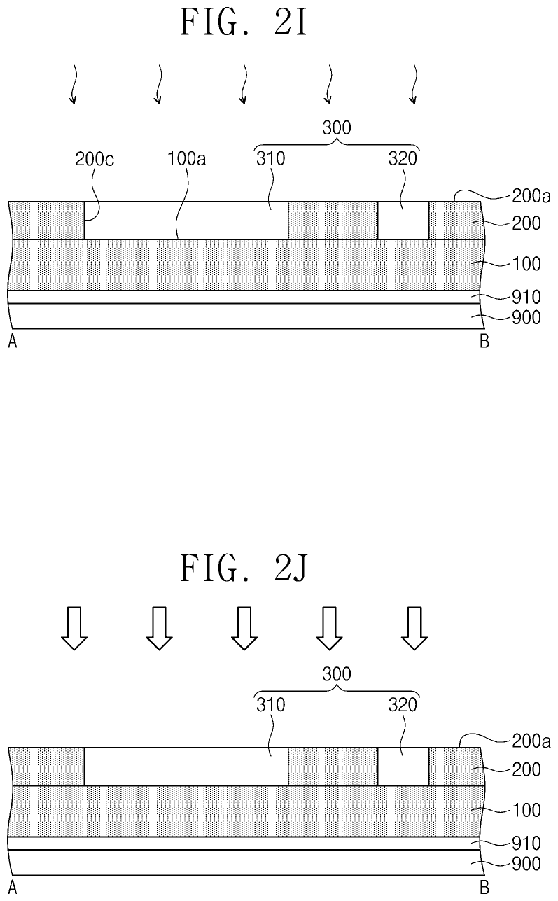

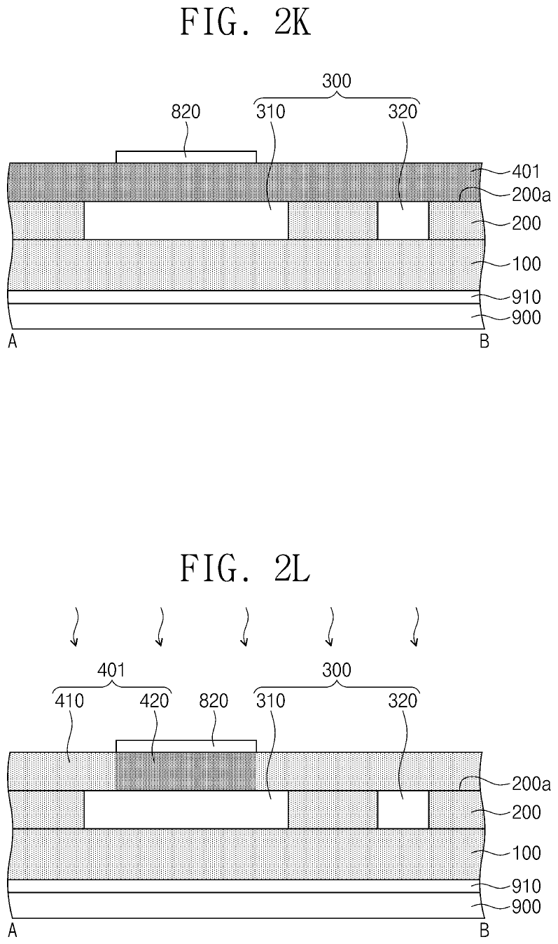

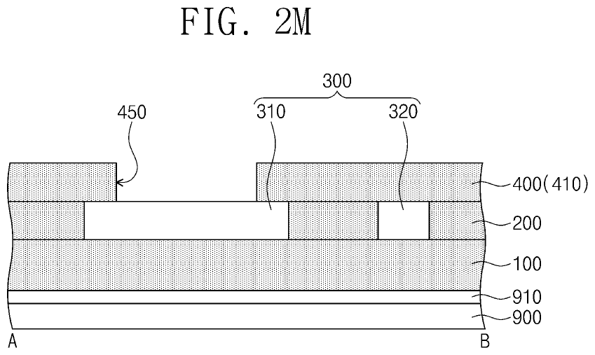

FIGS. 2A to 2M are views for explaining a method for fabricating an electrode sensor according to embodiments and, these views correspond to the cross-section taken along line A-B of FIG. 1A. Hereinafter, the description is made with reference to FIGS. 2A to 2M together with FIG. 1A, and description of the same features as in the above embodiments will be omitted. For the convenience, a plurality of polymers and a plurality of covalent bonds are described.

Referring to FIG. 2A, a preliminary organic substrate 101 and a sacrificial layer 910 may be formed on a temporary substrate 900. The temporary substrate 900 may include silicon, glass, and/or quartz. The sacrificial layer 910 may be formed on the top surface of the temporary substrate 900. The temporary substrate 900 may include metal or transparent conductive oxide. The metal may include, for example, copper (Cu), aluminum (Al), chromium (Cr), and/or titanium (Ti). The transparent conductive oxide may include indium-tin oxide ITO. A surface treatment process may be further performed on the top surface of the sacrificial layer 910. The surface treatment process may include an oxygen plasma treatment process as one example.

The preliminary organic substrate 101 may be formed on the top surface of the sacrificial layer 910. A bonding strength between an organic substrate 100 and the sacrificial layer 910 may be enhanced by the surface treatment process of the sacrificial layer 910. The forming of the preliminary organic substrate 101 may include: forming a preliminary layer by coating the sacrificial layer 910 with a polymer solution; drying the preliminary layer; and heat-treating the preliminary layer. The polymer solution may include a photo-initiator, a photocrosslinkable fluorine-containing polymer, and a solvent. The photocrosslinkable polymer may represent a polymer in which a crosslink bond has not been formed yet. The coating of the polymer solution may be performed by a spin coating process. The thickness of the preliminary organic substrate 101 may be determined by a composition ratio of the photo-initiator, the photocrosslinkable fluorine-containing polymer, and the solvent in a preliminary organic polymer solution. In another example, the thickness of the preliminary organic substrate 101 may be adjusted by a coating condition such as the number of revolutions in spin coating.

Referring to FIG. 2B, a first exposure process may be performed on the preliminary organic substrate 101. The first exposure process may be an ultraviolet irradiation process. Polymers within the preliminary organic substrate 101 are photocrosslinked with each other by the first exposure process, and thus the organic substrate 100 may be formed. Accordingly, the organic substrate 100 may include the photocrosslinked polymer. The strength and durability of the organic substrate 100 may be improved by the photocrosslink bond.

Referring to FIG. 2C, a first plasma treatment process may be performed on the organic substrate 100. The first plasma treatment process may be performed by using a plasma gas. The plasma gas may include an oxygen gas (O₂). In other examples, the plasma gas may include a nitrogen gas (N₂), an argon (Ar) gas, a helium (He) gas, and/or air. The first plasma treatment may include RF (radio frequency) plasma treatment. When plasma is applied to the fluorine-containing polymer, radicals may be formed on the fluorine-containing polymer. The radicals on the fluorine-containing polymer may become relatively stable in air. After the first plasma treatment process, the radicals may be formed on the top surface of the organic substrate 100. The organic substrate 100 includes the fluorine-containing polymer, and thus the radicals may become stable.

Referring to FIG. 2D, an organic insulating layer 201 may be formed on the organic substrate 100. The forming of the organic insulating layer 201 may include coating the top surface of the organic substrate 100 with a polymer solution. The polymer solution may include a photo-initiator, photocrosslinkable fluorine-containing polymers, and a solvent. The coating of the polymer solution may be performed by a spin coating process. The forming of the organic insulating layer 201 may further include performing a drying process and a heat treatment process. The drying process and the heat treatment process may be performed after the coating.

A first mask pattern 810 is formed on the organic insulating layer 201, and a portion of the top surface of the organic insulating layer 201 may be exposed by the first mask pattern 810. The first mask pattern 810 may be a photomask.

Referring to FIG. 2E, a second exposure process may be performed on the organic insulating layer 201. The second exposure process may be an ultraviolet irradiation process. The ultraviolet light may be emitted to a first portion 210 of the organic insulating layer 201 which is exposed by the first mask pattern 810. Due to the ultraviolet irradiation, the polymers of the first portion 210 of the organic insulating layer 201 may react with the radicals on the top surface of the organic substrate 100. Accordingly, covalent bonds may be formed between the first portion 210 of the organic insulating layer 201 and the organic substrate 100. The covalent bonds may be crosslink bonds. The organic insulating layer 201 may be strongly coupled to the organic substrate 100 due to the covalent bonds.

The polymers of the first portion 210 of the organic insulating layer 201 are crosslinked to each other by the ultraviolet irradiation, and thus the crosslinked polymers may be formed. Accordingly, the first portion 210 of the organic insulating layer 201 may have improved durability. According to embodiments, the forming of the covalent bonds between the organic substrate 100 and the first portion 210 of the organic insulating layer 201 and the forming of the crosslink bonds between the polymers of the first portion 210 of the organic insulating layer 201 may be performed by a single process. Accordingly, the fabrication of the electrode sensor may be simplified.

A second portion 220 of the organic insulating layer 201 may not be exposed to the ultraviolet light through the first mask pattern 810. A crosslink bond may not be formed between polymers of the second portion 220. Accordingly, after the second exposure process is completed, the first portion 210 of the organic insulating layer 201 may have a different chemical structure from the second portion 220. A crosslink bond may not be formed between the second portion 220 and the organic substrate 100. The coupling strength between the second portion 220 and the organic substrate 100 may be smaller than the coupling strength between the first portion 210 and the organic substrate 100.

Subsequently, the first mask pattern 810 may be removed.

After the second exposure process, a second heat treatment process may be further performed on the organic insulating layer 201. The second heat treatment process may be performed using substantially the same method and conditions as those described above in the first heat treatment process. Stress in the organic insulating layer 201 may be relieved by the second heat treatment. Accordingly, it is possible to prevent the organic insulating layer 201 and an organic insulating pattern 200 (FIGS. 2F to 2M) from being deformed (for example, contracted) during the fabrication process for the electrode sensor.

Referring to FIG. 2F, a development process is performed on the organic insulating layer 201, and thus the organic insulating pattern 200 may be formed. For example, the second portion 220 of the organic insulating layer 201 is removed by a developing solution, and thus a first opening 250 may be formed. A top surface 100a of the organic substrate 100 may be exposed by the first opening 250.

The first portion 210 of the organic insulating layer 201 includes the crosslinked polymer and thus may have a low reactivity with the developing solution. After the development process, the first portion 210 may remain. The organic insulating pattern 200 may correspond to the first portion 210 of the organic insulating layer 201. The organic insulating pattern 200 may have the first opening 250.

Referring to FIG. 2G, a second plasma treatment process may be performed on the organic insulating pattern 200 and the exposed organic substrate 100. The second plasma treatment process may be performed using the same conditions and method as those described above in the examples of the first plasma treatment process. During the second plasma treatment process, a top surface 200a of the organic insulating pattern 200, a side wall 200c of the organic insulating pattern 200, and the top surface 100a of the organic substrate 100 may be exposed to the plasma. The side wall 200c of the organic insulating pattern 200 may correspond to a side wall of the first opening 250, and the top surface 100a of the organic substrate 100 may correspond to the bottom surface of the first opening 250. Due to the second plasma treatment process, radicals may be formed on the top surface 200a of the organic insulating pattern 200, the side wall 200c of the organic insulating pattern 200, and the top surface 100a of the organic substrate 100.

Referring to FIG. 2H, a preliminary conductive layer 301 may be formed within the first opening 250. The forming of the preliminary conductive layer 301 may include filling the first opening 250 with a polymer solution through a printing process. In one example, the polymer solution may include insulating polymers (for example, PDMS) and conductive materials within the insulating polymers. The insulating polymers may be crosslinkable polymers. In another example, the polymer solution may include conductive polymers. The conductive polymers are crosslinkable. Unless otherwise specified, the crosslink in the present specification may include photocrosslink and heat-crosslink. The preliminary conductive layer 301 may not extend to the top surface 200a of the organic insulating pattern 200. The preliminary conductive layer 301 may be in physical contact with both the side wall 200c of the organic insulating pattern 200 and the top surface 100a of the organic substrate 100.

Referring to FIG. 2I, a third exposure treatment process is performed on the preliminary conductive layer 301, and thus a conductive layer 300 may be formed. The third exposure process may be an ultraviolet irradiation process. The polymers of the preliminary conductive layer 301 are crosslinked to each other by the ultraviolet irradiation, and thus the conductive layer 300 may be formed. Accordingly, the conductive layer 300 may include the crosslinked polymers. The conductive layer 300 may have improved durability.

The polymers of the preliminary conductive layer 301 may react with radicals, and these radicals may be radicals provided on the side wall 200c of the organic insulating pattern 200 and the top surface 100a of the organic substrate 100. Accordingly, covalent bonds may be formed both between the conductive layer 300 and the organic insulating pattern 200 and between the conductive layer 300 and the organic substrate 100. The covalent bonds may be crosslink bonds. The conductive layer 300 may be strongly coupled to both the organic substrate 100 and the organic insulating pattern 200 due to the covalent bonds.

The conductive layer 300 may include an electrode 310 and a wire 320. The electrode 310 and the wire 320 may be substantially the same as those illustrated above in FIGS. 1A and 1B.

In another example, the third exposure process may be omitted, and a third heat treatment process may be performed on the preliminary conductive layer 301. In this case, the polymers of the preliminary conductive layer 301 may include heat-crosslinkable polymers. The polymers of the preliminary conductive layer 301 are crosslinked to each other by the third heat treatment process, and thus the conductive layer 300 may be formed. The polymers of the preliminary conductive layer 301 may react with the radicals, and covalent bonds may be formed both between the conductive layer 300 and the organic insulating pattern 200 and between the conductive layer 300 and the organic substrate 100.

According to embodiments, the forming of the conductive layer 300 may be performed by the forming of the preliminary conductive pattern using the polymer solution and the exposure process with respect to the preliminary conductive pattern, and thus a separate etching process for patterning of electrode 310 and the wire 320 may be omitted. The etching process may include, for example, a reactive ion etching (RIE) process. Accordingly, separate etching equipment is not required during the fabrication process for the conductive layer 300, and thus the conductive layer 300 may be more easily fabricated. The fabrication process for the conductive layer 300 may be simplified, and fabrication process time for the conductive layer 300 may be reduced.

Referring to FIG. 2J, a third plasma treatment process may be performed on the organic insulating pattern 200 and the conductive layer 300. Due to the third plasma treatment process, radicals may be formed on the top surface 200a of the organic insulating pattern 200 and the top surface of the conductive layer 300. The third plasma treatment process may be performed using the same conditions and method as those described above in the examples of the first plasma treatment process.

Referring to FIG. 2K, a passivation layer 401 may be formed on the plasma-treated top surface 200a of the organic insulating pattern 200 and the plasma-treated top surface of the conductive layer 300. The forming of the passivation layer 401 may include coating the top surface of the organic insulating pattern 200 with a polymer solution. The polymer solution may include a photo-initiator and photocrosslinkable fluorine-containing polymers. Accordingly, the passivation layer 401 may include the photocrosslinkable fluorine-containing polymers. The coating of the polymer solution may be performed by a spin coating process. Subsequently, a drying process and a heat treatment process for the passivation layer 401 may be further performed.

A second mask pattern 820 may be formed on the passivation layer 401. The second mask pattern 820 may vertically overlap the electrode 310. The second mask pattern 820 may not vertically overlap the wire 320. The second mask pattern 820 may be a photomask.

Referring to FIG. 2L, a fourth exposure process may be performed on the passivation layer 401. The fourth exposure process may be an ultraviolet irradiation process. The ultraviolet light may be emitted to a first portion 410 of the passivation layer 401 which is exposed by the second mask pattern 820. The polymers of the first portion 410 may react with the radicals on the top surface 200a of the organic insulating pattern 200 and the radicals on the top surface of the conductive layer 300. Accordingly, covalent bonds may be formed both between the first portion 410 and the organic insulating pattern 200 and between the first portion 410 and the conductive layer 300. The covalent bonds may be crosslink bonds.

The polymers of the first portion 410 of the passivation layer 401 are crosslinked to each other by the ultraviolet irradiation, and thus the crosslinked polymers may be formed. Accordingly, the first portion 410 of the passivation layer 401 may have improved durability. A second portion 420 of the passivation layer 401 may not be exposed to the ultraviolet light through the second mask pattern 820, and thus a covalent bond may not be formed between the second portion 420 and the conductive layer 300. A crosslink bond may not be formed between polymers of the second portion 420. Accordingly, after the fourth exposure process is completed, the first portion 410 of the passivation layer 401 may have a different chemical structure from the second portion 420. Subsequently, the second mask pattern 820 may be removed.

According to embodiments, the forming of the covalent bonds between the first portion 410 of the passivation layer 401 and the organic insulating pattern 200 and the forming of the crosslink bonds between the polymers of the first portion 410 of the passivation layer 401 may be performed by a single process. Accordingly, the fabrication of the electrode sensor may be simplified.

After the fourth exposure process, a fourth heat treatment process may be further performed on the passivation layer 401. The fourth heat treatment process may be performed using substantially the same method and conditions as those described above in the first heat treatment process. Accordingly, it is possible to prevent the passivation layer 401 and the passivation pattern 400 (FIGS. 1B to 2M) from being deformed. Subsequently, the second mask pattern 820 may be removed.

Referring to FIG. 2M, a development process is performed on the passivation layer 401, and thus the passivation pattern 400 may be formed. The second portion 420 of the passivation layer 401 is removed by a developing solution, and thus a second opening 450 may be formed. At least a portion of the electrode 310 may be exposed by the second opening 450.

The first portion 410 of the passivation layer 401 includes the crosslinked polymer and thus may have a low reactivity with the developing solution. After the development process, the first portion 410 of the passivation layer 401 may remain. The passivation pattern 400 may correspond to the first portion 410. The passivation pattern 400 may have the second opening 450.

Referring to FIGS. 1A and 1B again, the temporary substrate 900 and the sacrificial layer 910 are removed, and thus the bottom surface of the organic substrate 100 may be exposed. According to an embodiment, the removing of the sacrificial layer 910 may be performed by an etching process where an etchant is used. According to another embodiment, the removing of the sacrificial layer 910 may be performed by using laser. The temporary substrate 900 may be separated from the organic substrate 100 together with the sacrificial layer 910. The fabricating of the electrode sensor 1 may be completed by the fabrication embodiments described above.

According to the present disclosure, the covalent bond may be provided between the organic substrate and the organic insulating pattern, between the organic substrate and the conductive layer, between the organic insulating pattern and the conductive layer, between the organic insulating pattern and the passivation pattern, and/or between the conductive layer and the passivation pattern. The durability of the electrode sensor may be enhanced by the covalent bonds.

According to the embodiments, the conductive layer includes the first polymer, and thus the conductive layer may have the improved durability and flexibility. The organic substrate, the organic insulating pattern, the conductive layer, and the passivation pattern are flexible, and thus the electrode sensor may become more flexible. Accordingly, the electrode sensor may be easily attached to the living body, and thus the electrode sensor may have great potential for application. The organic substrate, the organic insulating pattern, and the passivation pattern include the fluorine-containing polymer and thus may have high durability against chemical substances.

The above detailed description of the present disclosure is not intended to limit the embodiment of the inventive concept to the embodiments disclosed herein and may be used under various other combinations, changes, and environments without departing from the subject matters of the present disclosure. The following claims should be construed as including other embodiments.

What is claimed is:

1. An electrode sensor comprising:
an organic substrate comprising a polymer;
an organic insulating pattern disposed on the organic substrate and having an opening, the opening exposing the organic substrate;
a conductive layer disposed on the organic substrate and in the opening of the organic insulating pattern; and
a passivation pattern disposed on the organic insulating pattern and exposing a portion of the conductive layer,
wherein the conductive layer comprises a first polymer, and
the conductive layer is coupled to the organic substrate by a covalent bond.

2. The electrode sensor of claim 1, wherein the conductive layer is coupled to the organic insulating pattern by a covalent bond.

3. The electrode sensor of claim 1, wherein the organic insulating pattern is coupled to the organic substrate by a covalent bond.

4. The electrode sensor of claim 1, wherein the passivation pattern is coupled to the organic insulating pattern by a covalent bond.

5. The electrode sensor of claim 1, wherein the conductive layer is coupled to the passivation pattern by a covalent bond.

6. The electrode sensor of claim 1, wherein the conductive layer comprises an electrode and a wire, and the wire is connected to the electrode.

7. The electrode sensor of claim 6, wherein the passivation pattern covers a top surface of the wire,
wherein a top surface of the electrode is exposed by the passivation pattern.

8. The electrode sensor of claim 1, wherein the organic substrate comprises a fluorine-containing polymer, and
the organic insulating pattern comprises a fluorine-containing polymer.

9. The electrode sensor of claim 1, wherein the passivation pattern comprises a fluorine-containing polymer.

10. The electrode sensor of claim 1, wherein the conductive layer comprises a different material from the organic insulating pattern.

11. The electrode sensor of claim 1, wherein the first polymer comprises an insulating polymer,
the conductive layer further comprises a conductive material, and
the conductive material comprises a metal particle or a conductive carbon material.

12. The electrode sensor of claim 1, wherein the first polymer comprise a conductive polymer.

13. The electrode sensor of claim 1, wherein the organic substrate, the organic insulating pattern, the conductive layer, and the passivation pattern are flexible.

14. A method for fabricating an electrode sensor, the method comprising:
forming an organic insulating pattern on an organic substrate, the organic insulating pattern having an opening, a top surface of the organic substrate is exposed by the opening;
forming a preliminary conductive layer to cover the exposed top surface of the organic substrate, by filling the opening of the organic insulating pattern with a polymer solution; and
forming a conductive layer by performing an exposure process on the preliminary conductive layer and the organic insulating pattern,
wherein the conductive layer is coupled to the organic substrate by a covalent bond, and
the conductive layer comprises first polymers.

15. The method of claim 14, wherein the performing of the exposure process further comprises forming a covalent bond between the conductive layer and the organic insulating pattern.

16. The method of claim 14, further comprising performing a plasma treatment process on the exposed top surface of the organic substrate and a top surface of the organic insulating pattern to form a radical on the exposed top surface of the organic substrate,
wherein the forming of the preliminary conductive layer is performed after the plasma treatment process.

17. The method of claim 14, further comprising:
forming a passivation layer on the organic insulating pattern after the exposure process; and
irradiating the passivation layer with ultraviolet light to form a covalent bond between the passivation layer and the organic insulating pattern.

18. The method of claim 17, further comprising performing a plasma treatment process on the conductive layer and the organic insulating pattern to form radicals on a top surface of the conductive layer and a top surface of the organic insulating pattern,
wherein the forming of the passivation layer is performed after the plasma treatment process.

19. The method of claim 17, wherein the irradiating of the passivation layer with the ultraviolet light further comprises forming a covalent bond between the passivation layer and the conductive layer.

20. The method of claim 14, wherein the performing of the exposure process comprises forming photocrosslink bonds between the first polymers in the conductive layer.
